(19) United States
(12) Patent Application Publication
(10) Pub. No.: US 2004/0049221 A1
(43) Pub. Date: Mar. 11, 2004

(54) Method and apparatus for forming apertures in blood vessels

(75) Inventors: Amir Loshakove, Moshav-Bazra (IL); Ido Kilemnik, Herzelia (IL)

Correspondence Address: William H. Dippert, Esq., c/o Reed Smith LLP, 29th Floor, 599 Lexington Avenue, New York, NY, 10022-7650, US

(73) Assignee: By-Pass, Inc., Orangeburg, NY

(21) Appl. No.: 10/459,373

(22) Filed: Jun. 11, 2003

Related U.S. Application Data
(63) Continuation of application No. PCT/IL01/01019, filed on Nov. 4, 2001.
(63) Continuation-in-part of application No. 10/402,375, filed on Mar. 27, 2003.
Application No. 10/402,375 is a continuation of application No. PCT/IL01/00903, filed on Sep. 25, 2001.
(63) Continuation-in-part of application No. PCT/IL01/00600, filed on Jun. 28, 2001.
(63) Continuation-in-part of application No. 10/239,387, filed on May 30, 2003.
Application No. 10/239,387 is a 371 of international application No. PCT/IL01/00266, filed on Mar. 20, 2001.
(63) Continuation-in-part of application No. 10/239,364, filed on May 15, 2003.
Application No. 10/239,364 is a 371 of international application No. PCT/IL01/00267, filed on Mar. 20, 2001.
(63) Continuation-in-part of application No. 10/239,365, filed on Jun. 2, 2003.
Application No. 10/239,365 is a 371 of international application No. PCT/IL01/00074, filed on Jan. 25, 2001.
(63) Continuation-in-part of application No. 10/221,982, filed on Sep. 18, 2002.
Application No. 10/221,982 is a 371 of international application No. PCT/IL01/00069, filed on Jan. 24, 2001.
(63) Continuation-in-part of application No. 10/149,364, filed on Oct. 7, 2002.
Application No. 10/149,364 is a 371 of international application No. PCT/IL00/00609, filed on Sep. 8, 2000.
(63) Continuation-in-part of application No. 09/936,805, filed on Sep. 17, 2001.
Application No. 09/936,805 is a 371 of international application No. PCT/IB00/00310, filed on Mar. 20, 2000.
(63) Continuation-in-part of application No. 09/936,806, filed on Sep. 17, 2001.
Application No. 09/936,806 is a 371 of international application No. PCT/IL99/00670, filed on Dec. 8, 1999.
(63) Continuation-in-part of application No. 09/701,531, filed on Nov. 28, 2000.
Application No. 09/701,531 is a 371 of international application No. PCT/IL99/00284, filed on May 30, 1999.
(60) Provisional application No. 60/254,689, filed on Dec. 11, 2000.

(30) Foreign Application Priority Data
May 29, 1998 (IL) 124694
Mar. 19, 1999 (IL) 129067
Jun. 28, 2001 (IL) 144051

(51) Int. Cl. A61B 17/14

(52) U.S. Cl. 606/184

(57) ABSTRACT

Hole forming apparatus for forming an opening in a blood vessel, comprising:

a penetration head having a sharp tip adapted to be inserted through a wall of a blood vessel;

a base section defining an inner lumen;

at least one cutting surface defined on said base section and adapted to cut through a wall of said blood vessel without said apparatus pulling said blood vessel towards said cutting surface; and

a shaft mechanically coupling said penetration head to said base section,

said shaft defining a tissue holding shaft section between said base section and said penetration head, said penetration head adapted to prevent tissue transfixed on said shaft section from slipping off said penetration head,

said shaft having a first mechanically defined and axially locked resting position in which said penetration head is at least partially enclosed by said base section in said inner lumen and a second mechanically defined resting position in which said penetration head is axially locked relative to said base section and said shaft section is exposed between said base section and said penetration head.

### RELATED APPLICATIONS

The present application is related to U.S. provisional application No. 60/254,689 and to PCT publications and applications WO 99/62415, WO 00/56226, WO 00/56228, WO 01/70091, WO 01/70118, WO 01/70119, PCT/IL01/00266 and PCT/IL01/00600, the disclosures of which are incorporated herein by reference.

### FIELD OF THE INVENTION

The present invention relates to punches and similar devices for forming openings in blood vessels.

### BACKGROUND

Holes are formed in blood vessels for various reasons, principal among which are (a) for insertion of a tube (and later removing the tube sealing the hole); and (b) forming an anastomosis connection between a graft and the blood vessel.

PCT publication WO 00/74579, the disclosure of which is incorporated herein by reference, describes a hole former in which an outer tube is advanced and optionally rotated to cut into a blood vessel from the outside, while the cut part of the blood vessel is prevented from motion by a barb coupled to the hole former.

U.S. Pat. No. 5,129,913, the disclosure of which is incorporated herein by reference, describes a retracting shearing-cut punch, in which a non-rotating and blunt cutting head is inserted into a slit in a blood vessel and retracted while a base tube having a cutting lip is rotated. This effects a shearing cutting of a portion of the blood vessel as the cutting head is retracted towards and into the base tube.

### SUMMARY OF THE INVENTION

An object of some embodiments of the invention relates to methods for forming holes in blood vessels, using cutting action. Other embodiments possibly provide alternative or additional benefits.

In accordance with some exemplary embodiment of the invention, a hole former includes a penetration tip which optionally retracts after the tip is inserted through a blood vessel wall, a penetration head that passes through the wall and a base that does not pass through the wall. A cutting lip is provided on the base, to cut the vessel wall. Optionally, the cutting action is assisted by rotation of the base, for example complete and/or oscillatory rotations. Optionally, once some or all of the cutting is completed, the penetration head is retracted relative to the blood vessel, thus removing a plug that is cut out of the vessel. Optionally, the penetration head includes a thickened portion to prevent the plug from slipping off the head. Optionally, the retraction of the penetration head is relative to the base, for example the penetration head being spring loaded. Alternatively or additionally, the retraction is by retraction of the hole former as a whole, possibly advancing an over tube over the base to engage the opening formed in the vessel and prevent leakage.

It should be noted that in some embodiments of the invention, the hole former does not provide any contra. Rather, if any contra is necessary, it is provided by the target vessel itself. The penetration head is provided in these embodiments for preventing the cutting lip from slipping sideways and/or for preventing a cut out plug from falling into the blood vessel.

Optionally, the penetration head has a hollow lumen, which is optionally innerthreaded, barbed or otherwise treated to engage tissue. In an exemplary embodiment of the invention, the lumen is attached to a medicine reservoir inside or outside of the hole former. Alternatively or additionally, the penetration head is threaded on its outside, for example, to assist penetration.

In an alternative embodiment of the invention, cutting lips are provided on the penetration head alternatively or additionally to on the base. Alternatively or additionally to a cutting action, a shearing action is provided by the base and the head sliding by each other. Alternatively or additionally, anvil cutting action is provided by locating tissue between an anvil and a cutting edge. In some, but not all, embodiments, there is relative rotation between the head and the base. In an exemplary embodiment of the invention, the head is retracted towards the base to effect the cutting of a blood vessel from inside of the blood vessel.

An aspect of some embodiments of the invention relates to protecting an inner leaflet valve of a multi-tool anastomotic delivery system. In an exemplary embodiment of the invention, a same delivery system scaffold is used to deliver a hole former and to deliver an anastomotic connector (or for delivering a different tool). While replacing the two tools a valve is provided in the scaffold to prevent blood leakage from the vessel through the scaffold. In an exemplary embodiment of the invention, the hole former is inserted through the valve while covered while the hole former with a cover (e.g., a silicone tube), to prevent contact between sharp parts of the hole former and the valve. Optionally the cover is designed to be torn off, for example, being perforated and/or includes a rip cord.

An aspect of some embodiments of the invention relates to a hole former comprising a tube having a sharp cutting lip and a lumen in which there is provided means for engaging tissue, for example one way engaging, for example using barbs and/or an inner threading. As the tube is advanced (and/or rotated) against a blood vessel or other tissue, the tissue is cut by the lip and forced into the lumen, where it is engaged. Optionally, the tube comprises an outer threading, for example, to assist advancing into the tissue. Optionally, a central guide, for example a needle, is provided, to stabilize the location of the tube relative to the target tissue. Optionally, the central guide is threaded. The guide may be retractable relative to the tube or not. In different exemplary embodiments, the guide is advanced ahead of the lip, is approximately level with a plane defined by the lip or is retracted from the plane.

An aspect of some embodiments of the invention relates to a retracting hole former, in which the penetration head includes a cutting lip and the head rotates as it is retracted towards a base. Optionally, the base rotates. In an exemplary embodiment of the invention, the cutting lip fits inside the base. Alternatively, the cutting lip fits against the base.

An aspect of some embodiments of the invention relates to a hole former including a receptacle in a distal end of a penetration head for receiving a tissue plug being removed from a vessel wall during the formation of a hole in the vessel wall. Optionally, the receptacle is formed by a cutting lip formed on said penetration head. Alternatively or additionally, a cutting lip is formed on a base portion of said hole former. The cutting lip (one or both, if two) can be of various designs, for example, smooth, serrated and/or oblique. In an exemplary embodiment of the invention, the receptacle is deep enough to contain tissue plugs from one, two or more hole forming activities, even if the plug falls apart.

In an exemplary embodiment of the invention, the receptacle includes a plug extraction means. In one example, a spring element, for example a lump of soft silicon or a metal spring, is provided in the receptacle, so that when the hole forming is completed and the hole former removed from the vessel, the plug is ejected from the hole, at least partly, by the spring element. Alternatively or additionally, an axially retractable catch is provided in the receptacle, which is retracted, for example, manually or by a spring out of said receptacle and/or remains in place when said penetration head is moved away from said base.

An aspect of some embodiments of the invention relates to a hole former that combines anvil cutting and at least one of knife and shearing cutting for forming a hole in a blood vessel. In an exemplary embodiment of the invention, the anvil cutting is used to cut through an adventitsia of a blood vessel and the other cutting method is used for cutting through an intima of a blood vessel. The different cutting methods may be provided using a same cutting lip or using more than one cutting surface. In one example, an inclined part of the penetration head contacts the base to provide and anvil cutting action, while a cutting lip formed on the penetration head slides past the base to provide knife and/or shearing cutting action. In another example, the cutting lip provides knife cutting action until it contacts an inclined portion of the base and provides anvil cutting action.

An aspect of some embodiments of the invention relates to a rotating anvil-cutting hole former. Optionally, at least one of the anvil and the cutting head is spring-loaded so that when the anvil and head meet, one of them can retract, thus preventing and/or reducing damage to the cutting part. In an exemplary embodiment of the invention, the penetration head serves as a cutting part and the base is an anvil and is spring loaded. Optionally, the penetration head is retracted and rotated using a thread. Optionally the head can be rotated an infinite number of times once it reaches the base. Optionally, when the head reaches the base, it slips a thread, allowing the base to spring forward.

An aspect of some embodiments of the invention relates to anvil punching against a resilient material, which may be, for example, on the base or on the penetration head. Optionally, the cutting part of the hole former rotates relative to the anvil part. Optionally, when the penetration head is forcefully retracted, it pushes aside the resilient material and retracts into a predefined axial aperture in the anvil.

An aspect of some embodiments of the invention relates to designing hole former parameters. In an exemplary embodiment of the invention, D designates an outer diameter of a cutting lip, while d designates a minimum diameter of the hole former between the penetration head and the base. In an exemplary embodiment of the invention, the hole remover is designed to achieved a desired hole diameter. Generally, as D is closer to d, the amount of tissue removed by the hole forming operation tend to be smaller, as there is less room for the tissue plug to be contained in during the hole forming operation. While if D is substantially larger than d, a larger hole can be formed, having a diameter approaching and possibly passing D.

An aspect of some embodiments of the invention relates to various designs for a penetration tip and/or a penetration head. In an exemplary embodiment of the invention, the penetration head, which optionally serves as an anvil or as a plug holder for holding the vessel wall, is expandable, for example, as a spiral, as a deformable silicon element or as a plurality of radially extending (and, optionally, interconnected) arms. Alternatively, the penetration head may serve as a cutter, for example, in the spiral embodiment. Optionally, retraction of the penetration tip causes expansion of the penetration head.

Alternatively or additionally, an anvil is provided opposite only some of a circumference of a cutting lip.

In an alternative exemplary embodiment of the invention, the penetration tip and head comprise a threaded tube and the hole forming is performed by retracting the thread relative a base.

In an alternative exemplary embodiment of the invention, a penetration head includes a disk that is inserted on its side and/or in a distorted configuration into the vessel wall after the penetration tip enters the vessel. The disk is then used for the hole forming operation, for example, as an anvil.

In the examples of the threaded head and disk head, the cutting action may be, for example, knife, shearing and/or anvil, optionally utilizing a cutting lip on the penetration head.

In an exemplary embodiment of the invention, the penetration tip has the form of a one, two or more sided knife. Alternatively, the penetration tip has the form of a screw. Alternatively or additionally, the penetration head is deeply scalloped on one, two, three or more sides. Alternatively, the penetration head has a cross-section of a cross or a polygon, rather than having a circular cross-section as in some other embodiments.

In an alternative embodiment of the invention, one, two or more cutting spikes are formed as a cutting lip of the penetration head. The spikes have a wide base and a narrow tip and a cutting surface along their outer edge. In one example, two spikes are provided, with bases that together bridge the entire circumference of the penetration head.

An aspect of some embodiments of the invention relates to a needle-like hole former. In an exemplary embodiment of the invention, the base has the shape of a needle with an aperture, optionally oblique, at its tip. The needle itself may have, for example, a symmetric or an asymmetric conical tip. The edges of the aperture are sharpened. A tissue penetration tip is provided through the aperture and includes a trans-axial extension that has the general profile of the aperture. In use, the penetration tip is inserted into a blood vessel so that the trans-axial extension also passes through the blood vessel wall. The penetration tip is then retracted, pulling the blood vessel towards the base, so that the sharpened lips of the base and/or an optionally sharpened surface of the trans-axial extension cut the vessel wall.

An aspect of some embodiments of the invention relates to marking of punch motion. In an exemplary embodiment of the invention, the hole former includes a visual indication of the relative motion of the penetration head and the base and/or of the base relative to the rest of the hole former. In one example, a slot is formed in the base or an extension of the base, through which a marking on an extension of the penetration head is visible. Optionally, the hole former is provided via a delivery system. In an exemplary embodiment of the invention, the delivery system includes a window for viewing relative motion of the hole former and/or of other delivered tool, such as an anastomotic connector delivery tool, which optionally includes a similar progress indication. Optionally, reaching a desired point of progress is alternatively or additionally marked by a loud mechanical click.

An aspect of some embodiments of the invention relates to a side cutter for a blood vessel. In an exemplary embodiment of the invention, the side cutter includes an L shaped element having a sharpened tip. The tip is poked into a blood vessel and one arm of the L inserted into the blood vessel following the tip. The L element is optionally rotated so that its arm is parallel to the vessel axis. The L element is then retracted relative to a base, providing cutting action by an optional sharpened inner lip on the L and/or shearing action against the base. The base is optionally sharpened. The base may be provided on one sides of the L element or it may sandwich the L element. Optionally, the cutting arm of the L is parallel to the base, alternatively, the arm may be inclined towards the base or away from the base.

There is thus provided in accordance with an exemplary embodiment of the invention, hole forming apparatus for forming an opening in a blood vessel, comprising:

a penetration head adapted to be inserted through a wall of a blood vessel;

a base section;

at least one cutting surface defined on at least one of said penetration head and said base section and adapted to contact first an intima surface of said blood vessel; and

a shaft operatively connected to said cutting surface and configured to rotate said cutting surface relative to said blood vessel, while said penetration head and said base section are brought towards each other, to effect a removal of a plug tissue section from said blood vessel.

In an exemplary embodiment of the invention, said cutting surface is defined on said penetration head and wherein said shaft rotates said penetration head. Alternatively or additionally, said cutting surface is defined on said base section and wherein said shaft rotates said base section.

Alternatively or additionally, said plug is knife-cut by said cutting surface. Alternatively or additionally, said plug is formed by a shear-cut between said penetration head said base section. Alternatively or additionally, said plug is formed by an anvil-cut between said penetration head and said base section.

In an exemplary embodiment of the invention, said hole forming apparatus is adapted for insertion of said penetration head from outside a blood vessel.

Alternatively or additionally, said hole forming apparatus is adapted for insertion of said penetration head from inside a blood vessel.

Alternatively or additionally, said penetration head is adapted to pierce said blood vessel wall.

Alternatively or additionally, said cutting edge defines a tissue receptacle for holding said plug.

There is also provided in accordance with an exemplary embodiment of the invention, hole forming apparatus for forming an opening in a blood vessel, comprising:

a penetration head adapted to be inserted through a wall of a blood vessel;

a base section;

at least one cutting surface defined on at least one of said penetration head and said base section and defining a tissue receptacle for receiving a plug of said blood vessel wall; and

a shaft operatively connected to and configured to bring said penetration head and said base section and operative towards each other, to effect a removal of a plug from said blood vessel into said tissue receptacle.

In an exemplary embodiment of the invention, said cutting surface is defined on said penetration head. Optionally, said tissue receptacle is adapted to be retracted into said base section.

In an exemplary embodiment of the invention, said cutting surface is defined on said base section.

In an exemplary embodiment of the invention, the apparatus comprises a resilient element in said tissue receptacle that is compressed by said plug. Alternatively or additionally, the apparatus comprises a plug extractor mounted on said shaft and adapted to moved axially relative to said tissue receptacle to remove said plug.

There is also provided in accordance with an exemplary embodiment of the invention, hole forming apparatus for forming an opening in a blood vessel, comprising:

a penetration head adapted to be inserted through a wall of a blood vessel;

a base section;

a cutting surface defined on at least one of said penetration head and said base section;

an anvil surface defined on at least one of said penetration head and said base section; and

a shaft operatively connected to and configured to bring said penetration head and said base section towards each other, to effect a removal of a plug from said blood vessel by cutting action of said cutting surface and by anvil cutting action of said anvil surface.

In an exemplary embodiment of the invention, said cutting action comprises shearing cutting action between said cutting surface and said base section. Alternatively or additionally, said cutting action comprises knife cutting action by said cutting surface.

In an exemplary embodiment of the invention, said cutting surface takes part in said anvil cutting action. Alternatively, said anvil surface does not contact said cutting surface.

In an exemplary embodiment of the invention, at least one of said cutting surface and said anvil surface rotate.

In an exemplary embodiment of the invention, said anvil cutting action and said cutting action are applied to different layers of said blood vessel. Optionally, said anvil cutting action applies to an adventitsia layer of said blood vessel.

In an exemplary embodiment of the invention, said anvil cutting action comprises impulse anvil motion.

There is also provided in accordance with an exemplary embodiment of the invention, hole forming apparatus for forming an opening in a blood vessel, comprising:

a penetration head adapted to be inserted through a wall of a blood vessel;

a base section;

a cutting surface defined on at least one of said penetration head and said base section;

an anvil surface defined on at least one of said penetration head and said base section; and

a shaft operatively connected to and configured to bring said penetration head and said base section towards each other, to effect an anvil action between said cutting surface and said anvil surface, wherein at least one of said anvil surface and said cutting surface rotate relative to said blood vessel. Optionally, said rotation is mechanically synchronized to said bringing.

Alternatively or additionally, said anvil has a resilient backing that is deformed when said cutting surface contacts said anvil surface. Alternatively or additionally, said anvil springs back intermittently during said rotation. Alternatively or additionally, said rotation is mechanically unlimited in number of rotations. Alternatively or additionally, said anvil surface is inclined. Optionally, said inclined anvil surface shifts in a direction other than said rotation and said bringing towards each other when contacted by said cutting surface.

There is also provided in accordance with an exemplary embodiment of the invention, hole forming apparatus for forming an opening in a blood vessel, comprising:

a penetration head adapted to be inserted through a wall of a blood vessel;

a base section;

a cutting surface defined on at least one of said penetration head and said base section;

a resilient anvil portion having a surface defined on at least one of said penetration head and said base section; and

a shaft operatively connected to and operative to bring said penetration head and said base section towards each other, to effect an anvil action between said cutting surface and said anvil surface. Optionally, said anvil surface is resilient. Alternatively or additionally, said anvil portion is resiliently retractable.

There is also provided in accordance with an exemplary embodiment of the invention, a method of designing a hole forming apparatus for forming an opening in a blood vessel, comprising:

selecting a blood vessel punch design including an inner shaft having a diameter d and a cutting edge having a diameter D;

selecting a ratio between d and D and a value of D to effect a desired punched hole diameter. Optionally, the method comprises selecting a depth of a tissue receptacle portion of said punch design to affect said diameter.

There is also provided in accordance with an exemplary embodiment of the invention, a deformable hole former, comprising:

a base section; and

a deformable penetration head comprising a section formed of a resilient material, said head being adapted to be inserted through a wall of a blood vessel and adapted to deform to a configuration with a larger diameter after said penetration than during said penetrating; and

a shaft operatively connected to and operative to bring said penetration head and said base section towards each other, to effect a removal of a plug of tissue between said penetration head and said base section.

Optionally, the former comprises a retracting penetration tip adapted to deform said head when retracted.

There is also provided in accordance with an exemplary embodiment of the invention, a deformable hole former, comprising:

a base section; and

a deformable penetration head comprising at least one of a radially expanding spiral, a distorting disc and a plurality of radially extending arms, said head being adapted to be inserted through a wall of a blood vessel and adapted to deform to a configuration with a larger cross-section than during said penetrating; and

a shaft operatively connected to and operative to bring said penetration head and said base section towards each other, to effect a removal of a plug of tissue between said penetration head and said base section. Optionally, said disc is maintained in an axial orientation during said penetration.

There is also provided in accordance with an exemplary embodiment of the invention, hole forming apparatus for forming an opening in a blood vessel, comprising:

a threaded penetration head having a thread adapted to be inserted through a wall of a blood vessel;

a base section;

a shaft operatively connected to and operative to bring said penetration head and said base section towards each other, to effect a cutting motion by said thread. Optionally, said thread comprises a cutting edge. Alternatively or additionally, said thread cooperates with said base section to perform a shearing cutting action when they are brought together. Alternatively or additionally, said thread cooperates with said base section to perform an anvil cutting action.

There is also provided in accordance with an exemplary embodiment of the invention, hole forming apparatus for forming an opening in a blood vessel, comprising:

a needle defining an oblique aperture at its tip; and

a puller adapted to fit through said aperture and having a radially extending extension, said puller being adapted to engage a wall of said blood vessel towards the needle. Optionally, said oblique aperture defines a sharp cutting edge.

There is also provided in accordance with an exemplary embodiment of the invention, hole forming apparatus for forming an opening in a blood vessel, comprising:

two elements that cooperate to remove blood vessel tissue when moved towards each other;

a handle; and

a visual indicator embedded in said handle, that indicates a degree of relative motion of said elements.

There is also provided in accordance with an exemplary embodiment of the invention, an incision maker comprising:

an “L” shaped spike having a sharpened tip at the end of an arm thereof; and

at least one base surface generally parallel to said arm and adapted to perform a shearing cut against said arm.

Optionally, the incision maker comprises at least two generally parallel base surfaces. Alternatively or additionally, said base surface is not parallel to said arm.

There is also provided in accordance with an exemplary embodiment of the invention, hole forming apparatus for forming an opening in a blood vessel, comprising:

a penetration head having a sharp tip adapted to be inserted through a wall of a blood vessel;

a base section defining an inner lumen;

at least one cutting surface defined on said base section and adapted to cut through a wall of said blood vessel without said apparatus pulling said blood vessel towards said cutting surface; and

a shaft mechanically coupling said penetration head to said base section,

said shaft defining a tissue holding shaft section between said base section and said penetration head, said penetration head adapted to prevent tissue transfixed on said shaft section from slipping off said penetration head,

said shaft having a first mechanically defined and axially locked resting position in which said penetration head is at least partially enclosed by said base section in said inner lumen and a second mechanically defined resting position in which said penetration head is axially locked relative to said base section and said shaft section is exposed between said base section and said penetration head. Optionally, moving said shaft between said positions is not mechanically coupled to rotation of said base section. Alternatively or additionally, said penetration head has a geometry of a cone. Alternatively or additionally, said penetration head comprises at least one protrusion having a radial extent greater than a minimum diameter of said shaft section. Optionally, said at least one protrusion comprises a barb cut out of said shaft section. Optionally, said barb is elastic and flexible enough to be pushed against said shaft section by said wall of said vessel when said penetration head is inserted into said vessel.

Optionally, said penetration head comprises a retractable penetration tip. Alternatively or additionally, said penetration head is rotationally locked relative to said base section.

In an exemplary embodiment of the invention, in said first resting position said penetration head is fully retracted into said lumen. Alternatively or additionally, said shaft section is at least 150% greater than a width of a vessel for which the apparatus is designed.

In an exemplary embodiment of the invention, said apparatus is adapted for forming an aperture from outside a blood vessel.

Optionally, said shaft is spring loaded to move from said first resting position to said second resting position, when released. Alternatively or additionally, a motion of said shaft relative to said base is restricted to be between said two positions. Alternatively or additionally, said shaft has only two resting positions. Alternatively or additionally, said shaft is rigid.

In an exemplary embodiment of the invention, said inner lumen has a clearance relative to said penetration head such that no vascular tissue is cut between said penetration head and said inner lumen. Alternatively or additionally, said penetration head is adapted and arranged to not pull back said vessel wall during said cutting. Optionally, said penetration head defines a lumen in the direction of said wall.

In an exemplary embodiment of the invention, said apparatus comprises externally powered means from moving said shaft between said positions. Optionally, said apparatus comprises a pharmaceutical source coupled to an opening defined adjacent said penetration head and said base.

There is also provided in accordance with an exemplary embodiment of the invention, hole forming apparatus for forming an opening in a blood vessel, comprising:

a penetration head having a sharp tip adapted to be inserted through a wall of a blood vessel, said tip defining a lumen in a direction of said wall;

a base section defining an inner lumen;

at least one cutting surface defined on said base section and adapted to cut through a wall of said blood vessel; and

a shaft mechanically coupling said penetration head to said base section and fixing an axial position of said penetration head relative to said base. Optionally, said lumen elutes a pharmaceutical. Alternatively or additionally, said lumen is adapted to engage tissue. Alternatively or additionally, said lumen is adapted to allow one directional of motion of tissue relative to the lumen.

There is also provided in accordance with an exemplary embodiment of the invention, hole forming apparatus for forming an opening in a blood vessel, comprising:

a penetration head having a sharp tip adapted to be inserted through a wall of a blood vessel;

a base section defining an inner lumen;

at least one cutting surface defined on said base section and adapted to cut through a wall of said blood vessel without said apparatus pulling said blood vessel towards said cutting surface; and

a shaft mechanically coupling said penetration head to said base section and fixing an axial position of said penetration head relative to said base. Optionally, said inner lumen is adapted to engage tissue. Alternatively or additionally, said penetration head has a fixed diameter.

There is also provided in accordance with an exemplary embodiment of the invention, a method of inserting a tool into a scaffold having an inner valve, comprising:

covering at least one sharp edge of said tool with a cover;

inserting said tool through said valve; and

removing said cover. Optionally, said removing comprises tearing.

There is also provided in accordance with an exemplary embodiment of the invention, hole forming apparatus for forming an opening in a blood vessel, comprising:

a base section defining an inner lumen, said inner lumen being adapted to engage blood vessel tissue; and

at least one cutting surface defined on said base section and adapted to cut through a wall of said blood vessel. Optionally, said adaptation comprises an inner threading. Alternatively or additionally, said apparatus comprises a trans-axial stabilizer having a fixed axial position relative to said cutting surface.

There is also provided in accordance with an exemplary embodiment of the invention, hole forming apparatus for forming an opening in a blood vessel, comprising:

means for forming an aperture in a blood vessel; and

eluting means for providing a pharmaceutical at said aperture. Optionally, said means for forming comprises a cutting means. Alternatively or additionally, said means for forming comprises a shearing means. Alternatively or additionally, said means for forming comprises anvil cutting means.

There is also provided in accordance with an exemplary embodiment of the invention, a method of forming an aperture in a blood vessel, comprising:

inserting a penetration head into a wall of a blood vessel; and

advancing a cutting base against said blood vessel while not applying a contra force to said blood vessel via said penetration head. Optionally, advancing comprises advancing using rotational motion.

### BRIEF DESCRIPTION OF THE FIGURES

Non-limiting embodiments of the invention will be described with reference to the following description of exemplary embodiments, in conjunction with the figures. The figures are generally not shown to scale and any measurements are only meant to be exemplary and not necessarily limiting. In the figures, identical structures, elements or parts which appear in more than one figure are preferably labeled with a same or similar number in all the figures in which they appear, in which:

FIG. 1A illustrates a hole former having an outer cutting lip, in accordance with an exemplary embodiment of the invention;

FIG. 1B illustrates a hole former having an inner cutting lip, in accordance with an exemplary embodiment of the invention;

FIGS. 2A-2E are cut-through views of an exemplary hole former, in accordance with an exemplary embodiment of the invention;

FIG. 3 illustrates various dimensions of a penetration head that may be relevant in accordance with an exemplary embodiment of the invention;

FIGS. 4A and 4B illustrate plug removal mechanisms in accordance with an exemplary embodiment of the invention;

FIG. 5 illustrates a base retraction mechanism, in accordance with an exemplary embodiment of the invention;

FIG. 6 illustrates an alternative hole former, in accordance with an exemplary embodiment of the invention;

FIGS. 7A-7I illustrate various penetration tip and penetration head designs, in accordance with exemplary embodiments of the invention;

FIGS. 8A and 8B illustrate an expanding penetration head, in accordance with an exemplary embodiment of the invention;

FIGS. 9A and 9B illustrate an alternative expanding penetration head, in accordance with an exemplary embodiment of the invention;

FIGS. 10A and 10B illustrate another alternative expanding penetration head, in accordance with an exemplary embodiment of the invention;

FIGS. 11A and 11B illustrate a geometry changing anvil, in accordance with an exemplary embodiment of the invention;

FIG. 12 illustrates a resilient anvil hole former, in accordance with an exemplary embodiment of the invention;

FIG. 13 illustrates a thread-type penetration head, in accordance with an exemplary embodiment of the invention;

FIGS. 14A and 14B illustrate a needle-type hole former, in accordance with an exemplary embodiment of the invention;

FIGS. 15A and 15B illustrate two variants of an incision maker, in accordance with an exemplary embodiment of the invention;

FIGS. 16A and 16B illustrate a hole former in accordance with an alternative embodiment of the invention;

FIGS. 17A-17E illustrate the use of the hole former of FIG. 16, in accordance with an exemplary embodiment of the invention; and

FIG. 18 illustrates a tip of a hole former in accordance with an alternative embodiment of the invention.

### DETAILED DESCRIPTION OF EXEMPLARY EMBODIMENTS

Side to end anastomosis connections typically require an opening to be made in the “side” vessel, which is typically a target vessel. If an incision is made in the side vessel, expanding the incision to an elliptical or circular opening, as typically required in an anastomosis connection, may cause tearing and/or distortion of the target vessel. An alternative method is to punch or cut out a hole in the vessel (e.g., using the methods described in the background). However, the inventors have found that such punching may create a hole with one or more tears on its circumference. For example, punching a 2.5 mm diameter hole in an aorta, typically causes a tear, which, once the anastomosis is completed, may expand and cause a leak. In some cases, the size of the hole in the aorta has been shown to affect the probability of causing a tear, however, a minimal hole size may be required in order to prevent distortion of the aorta when performing an anastomosis of a larger diameter.

A blood vessel is formed of several layers. The outermost layer is a tough fibrous layer called the adventitsia. The innermost layer is called the intima. The inventors have found that if the cutting proceeds from the outside in, the adventitsia may catch on the cutting element and distort the intima before it is cut. In addition, the inventors have determined that different cutting methods may be useful for the different layers of the blood vessel.

Once a portion (a plug) is cut out of the vessel wall, it is typically desirable to prevent the plug from falling into the blood flow. In addition, the plug may fall apart during or after the hole formation.

One or more of the above problems is solved by some of the embodiments of the invention.

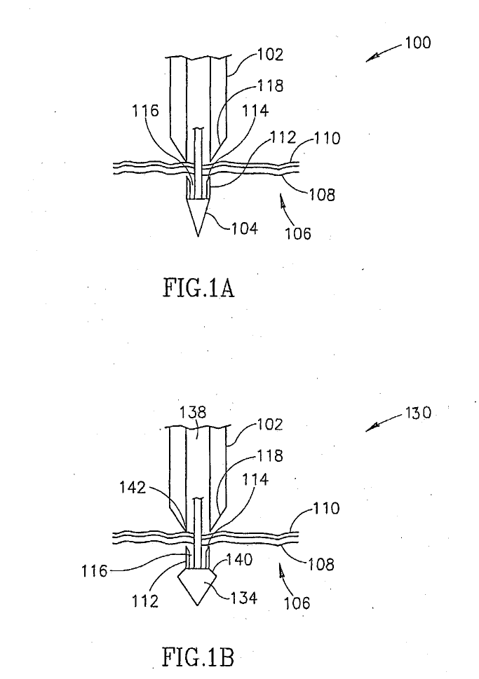

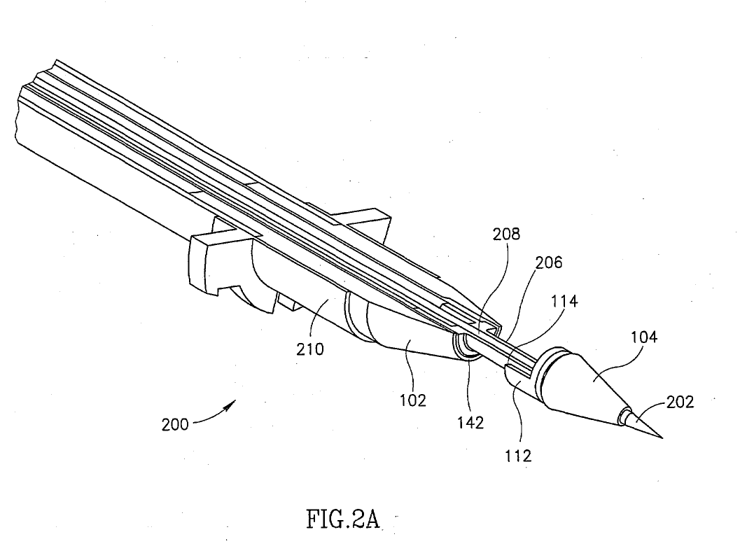

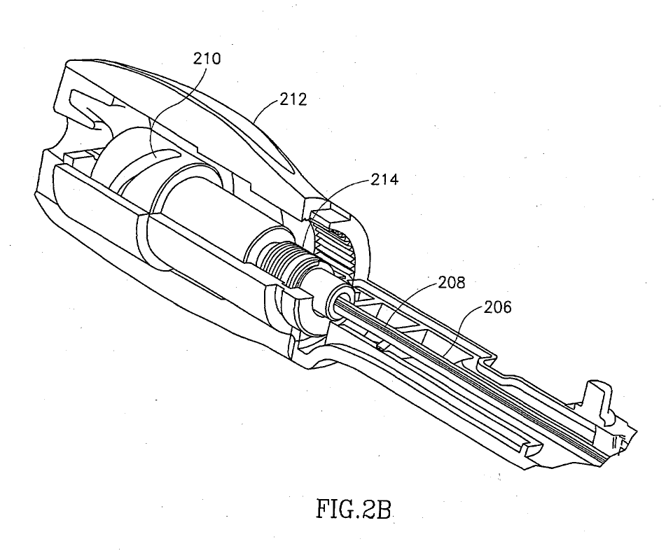

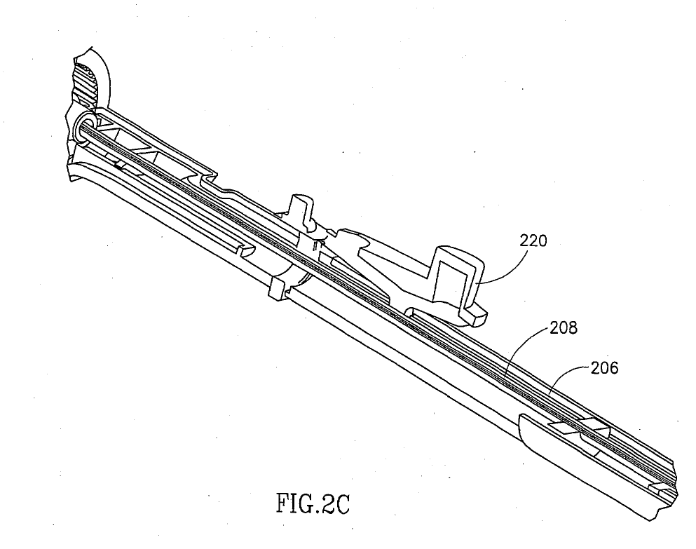

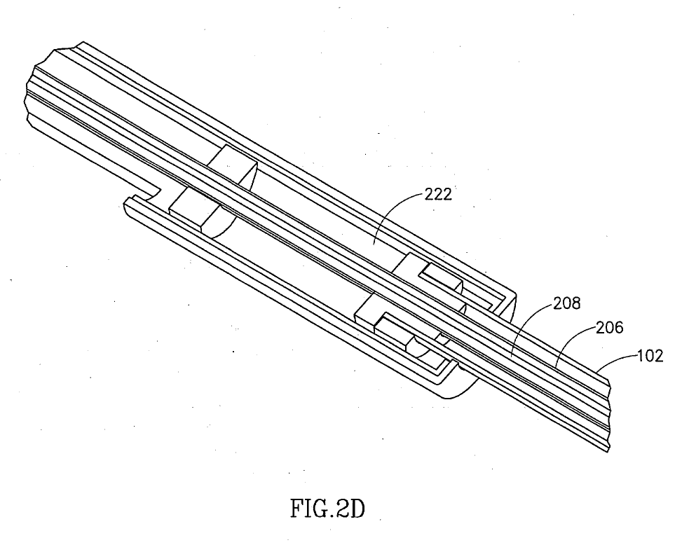

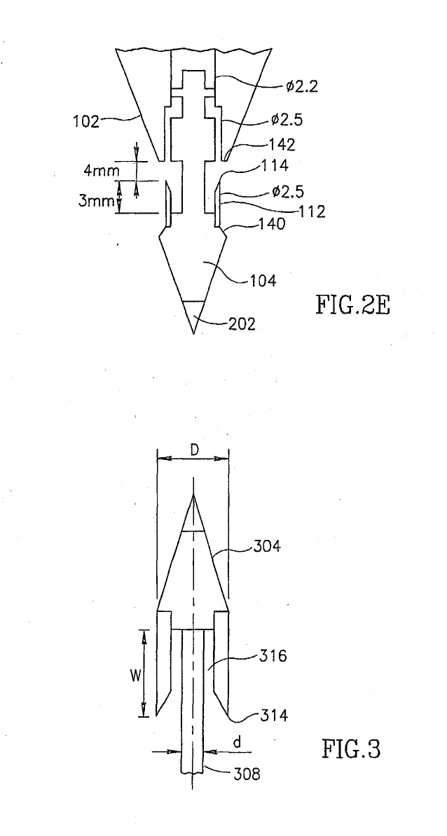

FIG. 1A illustrates a hole former 100 in accordance with an exemplary embodiment of the invention, comprising a base tube 102 and a penetration head 104 for insertion through a wall of a blood vessel 106. As shown, vessel 106 comprises an intima layer 108 and an adventitsia layer 110. As shown for example in FIG. 2, the tip of penetration head 104 may comprises a retractable penetration tip.

In an exemplary embodiment of the invention, penetration head 104 comprises a cutting lip 114 that cuts into vessel 106 when retracted towards the vessel. Optionally, cutting lip 114 is formed as the rim of a cup 116 having a wall 112. Cup 116 desirably serves to contain a tissue plug that is cut out of vessel 106 by cutting lip 114.

In the embodiment of FIG. 1A, base tube 102 defines an anvil surface 118 that contacts cutting lip 114 when penetration head 104 is retracted sufficiently. In an exemplary embodiment of the invention, as it is retracted, lip 114 performs a knife cutting action until it nears anvil 118, where it performs an anvil cutting action, which may be suitable for cutting through adventitsia 110.

FIG. 1B shows an alternative hole former 130, in which the knife cutting action and the anvil action are performed by different surfaces. Wall 112 has an outer diameter smaller than an inner diameter of base tube 102, so that cup 116 can be retracted into a bore 138 of tube 102. If the clearance between lip 114 and bore 138 is small enough, a shearing cutting action can be performed between penetration head 104 and base tube 102. Optionally, lip 114 is sharp enough for performing a knife cutting action.

In an exemplary embodiment of the invention, anvil cutting is provided between a cutting lip 142 of base tube 102 and an anvil portion 140, optionally inclined, of penetration head 134.

Optionally, one or both of penetration head 104 and base tube 102 rotate, in same or in opposite directions. Alternatively to complete rotations, oscillatory rotation is provided.

When retracting penetration head towards base tube 102, one or both of head 104 and tube 102 may be moved. Optionally, for example as described below, the motion is intermittent, allowing an impulse anvil cutting action to be achieved.

Coupling between advancing and rotation is optional. In one example, coupling is achieved by a threading that links advancing to rotation. Alternatively to rotation during retraction, rotation is performed after retraction (e.g., when the edges begin to pinch the vessel wall). Optionally, rotation and retraction are controlled separately, for example using one control for rotation and one for retraction.

FIGS. 2A-2E are cut-through views of an exemplary hole former 200, in accordance with an exemplary embodiment of the invention and similar to the embodiment of FIG. 1B.

FIG. 2A shows an optional retracting penetration tip 202 that is retracted by retracting a shaft 208 to which it is attached after penetration, so that the sharp tip does not damage the far wall of the blood vessel. Optionally, the retraction of the tip unlocks a retraction mechanism that manually or automatically (e.g., using a spring or a motor) retracts the penetration head towards the base section. Also shown is a shaft 206 used for retracting penetration head 104. Former 200 is shown mounted in a delivery system 210, optionally a split delivery system.

FIG. 2B shows a handle section of former 200, which comprises, for example, a rotating handle 212. A slot 210 is used to guide the retraction of penetration tip 202 once the tip penetrates a blood vessel. A threading 214 is used, for example, to control the retraction and rotation of penetration head 104 during use of hole former 200.

FIG. 2C shows a central section of former 200, including an optional clip 220 for locking former 200 into delivery system 210.

FIG. 2D shows a section of former 200 in which base tube 102 is coupled to the rest of former 200. As will be shown below, an optional volume 222 is used to contain a resilient element (e.g., silicon or a spring) that couples base tube 102 to former 200.

FIG. 2E shows exemplary measurements for system 200 for use in a human aorta.

It should be noted that, in an exemplary embodiment of the invention, once the plug is removed from the vessel wall, base tube 102 is advanced into the formed hole, for example, to prevent blood leakage.

FIG. 3 illustrates various dimensions of a penetration head 304 that may be relevant in accordance with an exemplary embodiment of the invention. A diameter d is the outer diameter of a shaft 308, used to retract head 304. A diameter D is the outer diameter defined by a cutting lip 314. A depth W is a depth of a tissue receptacle area 316 that contains the plug. The inventors have determined that the size of tissue plug removed from the target vessel is dependent on the geometry of the tissue receptacle. Thus, if W is too small, the tissue plug will be restricted in size. Similarly, if D is near d, there is less room for the tissue plug. Optionally, the use of a cutting lip 314 rather than a blunt end ensures that less tissue will slip past, since lip 314 cuts into the tissue and holds it in place. Optionally, the receptacle geometry is designed to affect a certain plug geometry. For example, if the receptacle fills up before cutting is completed, the plug diameter will decrease. The direction of decrease along the thickness of the plug may depend on the direction of cutting and/or receptacle orientation. For example, if the tissue receptacle and/or cutting lips are formed on tube 102, the decrease will be towards the blood vessel. In addition, knife cuts may be used to ensure that earlier cut tissue will have a known diameter, while a shearing cut can be used to ensure that later cut tissue will have a geometry based on available receptacle volume. An hourglass profile may be achieved by cutting from both sides of the vessel towards the middle, while using a limited volume tissue receptacle defined between the two cutting sides.

Various rotation/axial ratios may be used, for example, 1/1—one rotation per mm advance. In one example, at least 10 or at least 30 rotations are provided during a hole forming. In another example, only one, or fewer rotations are provided.

If W is large enough, the tissue plug removed from the body will lodge in receptacle 316 and additional use of the hole forming system will be difficult. In particular, a smooth cutting action may indicate a large value for W, so that the tissue plug is substantially inaccessible form outside. In an exemplary embodiment of the invention, mechanisms to assist in removing the plug are provided.

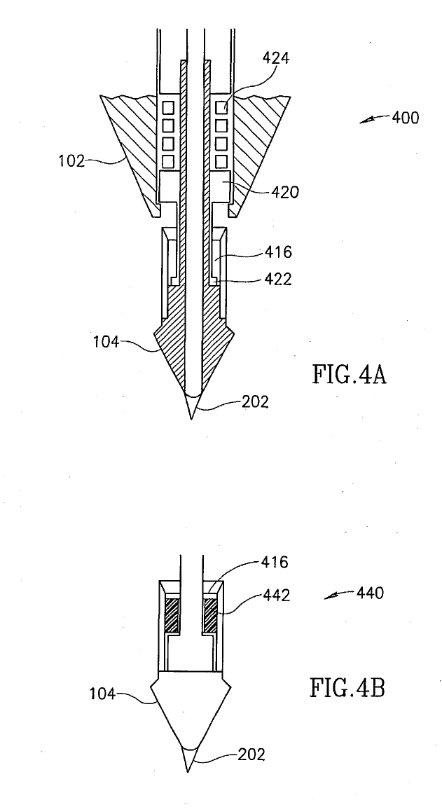

FIG. 4A shows a penetration head 400 in which a tissue extractor 420 is provided for pulling a tissue plug out of a tissue receptacle 416. In an exemplary embodiment of the invention, extractor 420 includes one or more radial extensions (or a lip) 422 that lie inside receptacle 416. When penetration head 104 is advanced, the tissue plug catches on extensions 422 and is extracted from receptacle 416. An optional resilient element 424, for example a spring a soft rubber is provided to allow tissue retractor 420 to be pushed towards base 102. In an alternative embodiment, retractor 420 is free-moving.

FIG. 4B shows an alternative mechanism 440, in which a resilient element 442, such as a spring or a silicon plug is provided in tissue receptacle 416. The resilient element is compressed by the plug during the hole forming operation and rebounds when the operation is complete, to urge out the plug.

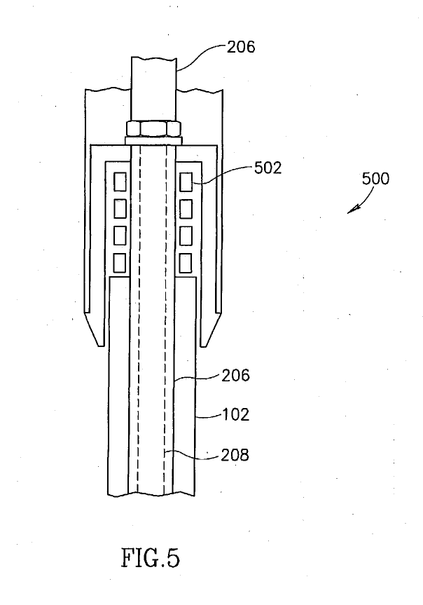

In some embodiments of the invention, for example as shown in FIG. 1A, a cutting lip contacts a non-moving element, and may be damaged thereby. FIG. 5 illustrates a base retraction mechanism 500, in accordance with an exemplary embodiment of the invention, which allows base 102 to resiliently retract. Thus, for example, when contacted by cutting lips, base 102 is pushed back by the lips instead of the lips being ground down. One potential advantage of such resilient contact is that it allows a looser manufacturing tolerance when designing a thread for coupling axial and rotational motion of penetration head 104.

In an exemplary embodiment of the invention, mechanism 500 comprises a resilient element 502 (or base 102 may be made resilient) such as a lump of soft silicon rubber or a spring, that allows some axial motion of base 102.

An additional potential advantage of such resilience is that it allows penetration head 104 to continue rotating after it contacts base 102. An additional potential advantage is that if penetration head jumps a thread after it contacts base 102, this causes an impulse motion of head 104 relative to base 102, which may assist in cutting the adventitsia.

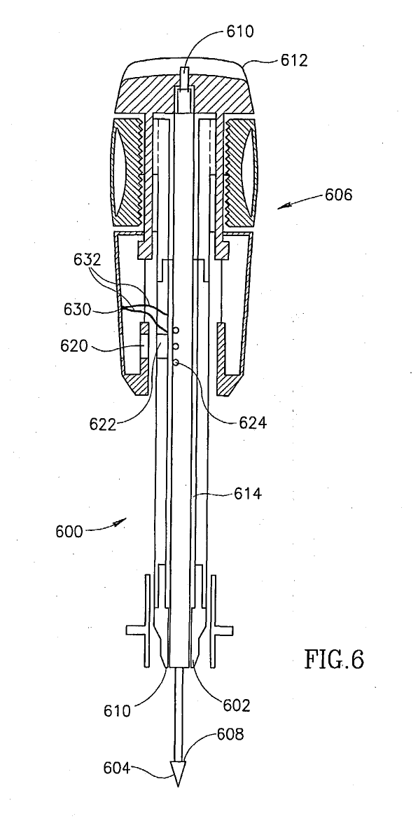

FIG. 6 illustrates an alternative hole former 600, in accordance with an exemplary embodiment of the invention. In this embodiment, former 600 comprises a penetration head 604 with an optional retracting penetration tip (not shown). Slicing action is optionally provided between the upper edge of penetration head 604 and the inner diameter of a base 602. Alternatively or additionally, knife cutting action is provided by an inner lip 608 of penetration head and/or a forward lip 610 of base 602. One or both of head 604 and base 602 rotate. Optionally, head 604 is retracted using a threaded drive actuated in handle 606. Alternatively, head 604 (and similarly heads on other embodiments described herein) may be retracted using a spring loaded mechanism.

Also useful, as illustrated for example, in FIG. 6, are various marking systems for indicating the progress of hole forming. One exemplary system comprises an aperture (or transparent portion) 620 defined in handle 606 and a second aperture 622 formed in base 602. One or more visual markings 624 on a shaft 614 that is coupled to penetration head 604 may be visible through the apertures/transparent sections to indicate a relative location of penetration head 604 and base 602.

Another exemplary indication system comprises a transparent dome 612 through which is visible the extension of a bar 610 (which extends as penetration head 604 is retracted), is visible.

Another exemplary system is an electrical system in which references 624 indicate contacts (rather than markings) on shaft 614 short together leads 632 to allow a battery (not shown) to power light 630, a LED for example. This allows the indication to be better located than using mechanical means. Alternatively or additionally, a mechanical (or electrical) sound, such as a click is sounded when the retraction of head 604 is completed. Possibly, different sounds are generated during retraction and after head 604 contacts base 602. Alternatively or additionally, a resistor and slide arrangement is used to indicate progress on a meter other suitable scale display.

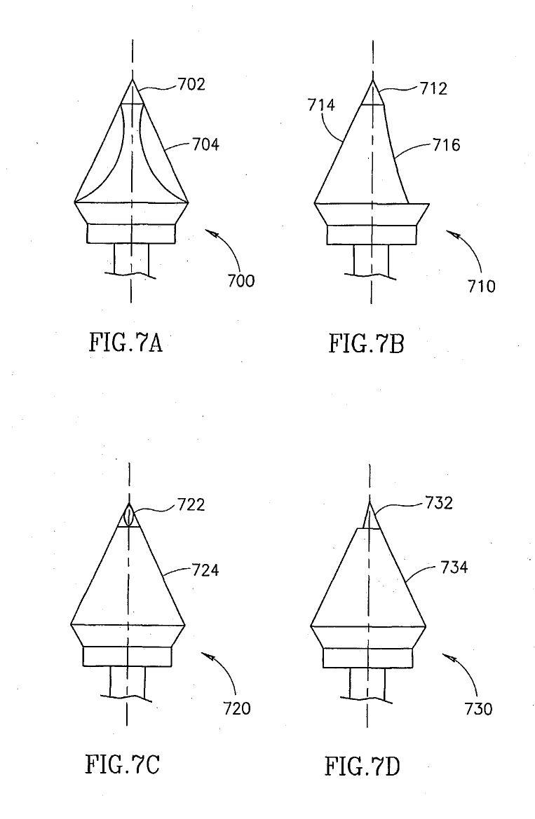

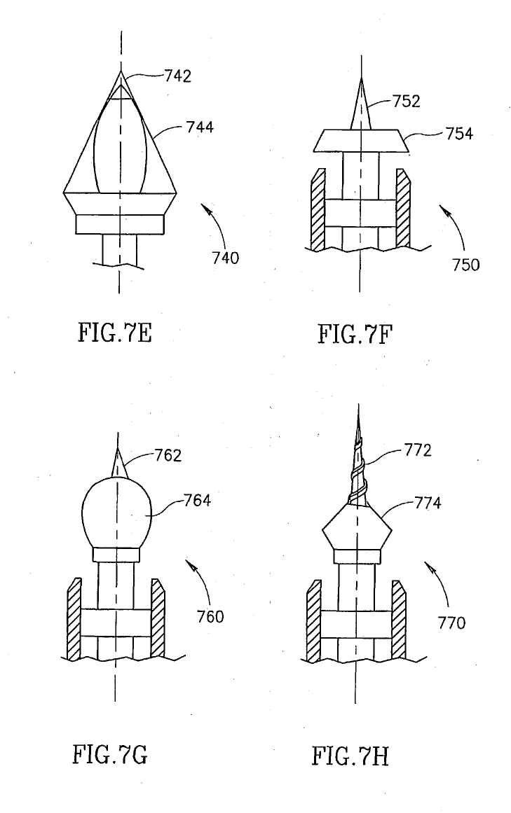

FIGS. 7A-7I illustrate various penetration tip and penetration head designs, in accordance with exemplary embodiments of the invention. The penetration tips are optionally retractable in each of the diagrams shown.

FIG. 7A shows a penetration head 700 including a head body 704 that is deeply scalloped on one, two, three or more sides and a penetration tip 702, that is conical.

FIG. 7B shows a penetration head 710 including a head body 714 that is asymmetric and sharpened along one edge 716 thereof and having a matching knife shaped penetration tip 712.

FIG. 7C shows a penetration head 720 including a conical head body 724 and a penetration tip 722, that is scalloped.

FIG. 7D shows a penetration head 730 including a conical head body 734 and a penetration tip 732, that is a one sided knife.

FIG. 7E shows a penetration head 740 in which scalloping on a head body 744 matches scalloping on a penetration tip 742.

FIG. 7F shows a penetration head 750 in which a head body 754 is a truncated cone having a longer and sharper penetration tip 752, for example, having a length that is 2 or three times its diameter.

FIG. 7G shows a penetration head 760 in which a head body 764 is bulbous and blunt, with a regular penetration tip 762.

FIG. 7H shows a penetration head 770 in which a head body 774 is associated with a threaded penetration tip 772 that is optionally rotated as it is advanced.

FIG. 7I shows a penetration head 780 in which a head body 784 and its associated penetration tip 782 are formed in the shape of a knife having the cross-section of a cross.

Other variations are contemplated as well, for example, one or both of the cutting lips on the penetration head and base 102 may be oblique relative to the axis or relative to the radius of the system (e.g., have a non-constant radius). Such oblique elements may be provided, for example, for embodiments with inner lip cutting or with outer lip cutting. The different parts may have different degrees of obliqueness.

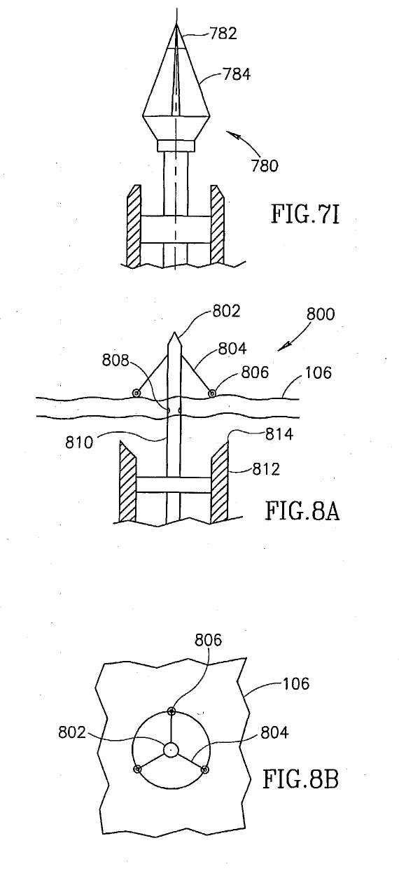

FIGS. 8A and 8B illustrate an expanding penetration head 800, in accordance with an exemplary embodiment of the invention. Head 800 comprises a penetration tip 802 mounted on a shaft 810. A plurality of arms 804 extend radially at an angle from shaft 810. Optionally, the arms are contained in slots 808 defined in shaft 810. In an exemplary embodiment of the invention, the arms spring out when shaft 810 exits a confining outer base tube 812 and after it passes through the confinement of a wall of vessel 106. In an exemplary embodiment of the invention, arms 804 end in rounded tips 806. FIG. 8B shows a top view of FIG. 8A. Optionally, arms 804 are slivers formed out of the body of shaft 810.

In use, shaft 802 is retracted relative to base portion 812. Cutting action may be achieved by a cutting edge 814 of tube 812. Alternatively or additionally, tips 806 serve as a partial anvil for urging tissue against cutting edge 814. Optionally, shaft 802 and/or base 812 are rotated.

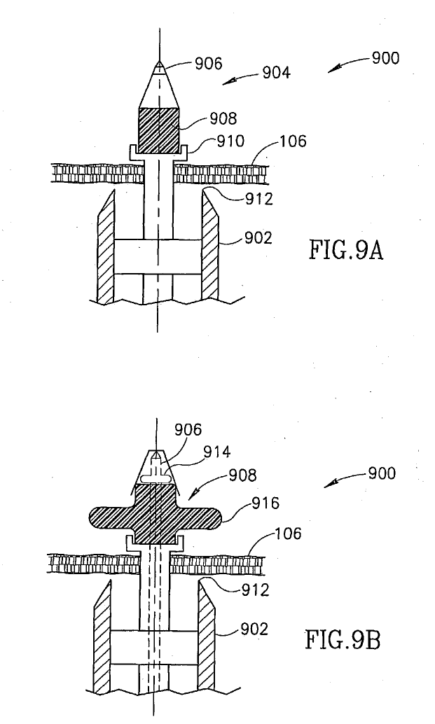

FIGS. 9A and 9B illustrate an alternative expanding penetration head 904, in accordance with an exemplary embodiment of the invention. A hole former system 900 comprises a base tube 902 having a cutting edge 912 and an expanding head that has a small diameter when inserted through a vessel 106 (FIG. 9A) and a larger diameter during hole forming (FIG. 9B). In an exemplary embodiment of the invention, head 904 comprises a resilient and/or expandable element 908, for example comprising silicon or other fluid or semi-fluid material, that is deformed and caused to expand out so that extensions 916 (or a disc) are formed. In an exemplary embodiment of the invention, A penetration tip 906 of head 904 (and optionally an associated base 914) or the whole of head 904 are retracted relative to a base portion 910 of head 904, this causes the silicon element 908 to be axially compressed and radially extend. Alternatively, element 908 may be expanded or it may be deformed by the advancement of a rod into the element from the direction of tube 902.

In an exemplary embodiment of the invention, extensions 916 serve to urge the wall of vessel 106 towards base 902. Alternatively or additionally, extensions 916 serve as an anvil for cutting edge 912. Optionally, silicon element 908 has one or more hard patches on its surface. In an exemplary embodiment of the invention, such hard patches can be used for the anvil cutting action, however, they are not required. Alternatively or additionally, extensions 916 fit inside base tube 902 and provide for shearing cutting action. Alternatively or additionally, the expansion of element 908 causes one or more sharp spikes or cutting edges (not shown) to extend in the direction of base 102. Optionally, extensions 916 are inclined at the point of contact with cutting edge 912, providing for an angular anvil cutting action. Optionally, the resilience of element 908 is such that when cutting edge 912 meets/nears extensions 916, the extensions give, allowing a sliding of edge 912 relative to extensions 916.

It should be noted that even a soft anvil or scissors part can provide some benefits over a free cutting action. In addition, the resiliency of the silicon can be manipulated (during manufacture) to provide a maximum hardness that still allows the silicon to be deformed.

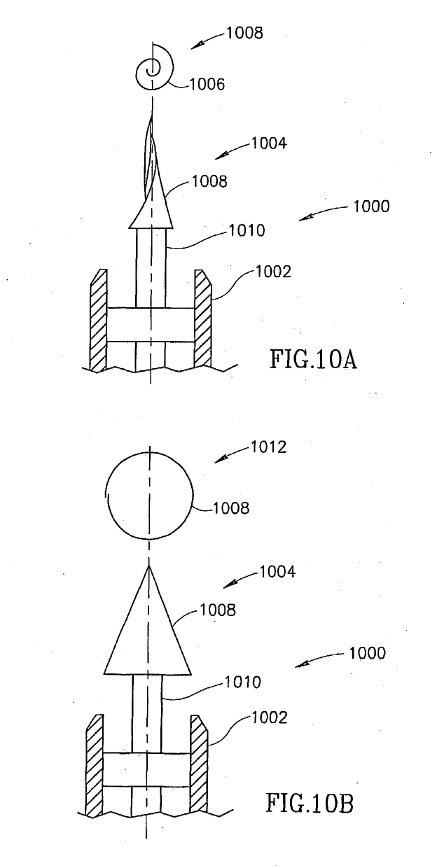

FIGS. 10A and 10B illustrate a hole former 1000 that includes an expanding penetration head 1004, in accordance with an exemplary embodiment of the invention.

In an exemplary embodiment of the invention, head 1004 comprises a thin sheet 1008 that is tightly wound around its axis, as shown in a cross-section 1006. FIG. 10B shows former 1000 after deployment, when head 1004 is released to achieve a conical shape. A cross-section is shown as reference 1012. A shaft 1010 is optionally welded to the side or to the tip of head 1004. Alternatively, sheet 1008 is manufactured out of shaft 1010.

Once head 1004 expands, head 1004 may be retracted towards a base tube 1002 to provide for cutting action, for example, knife, shearing and/or anvil cutting action, as described herein, depending, inter alia, on the relative geometry of head 1004 and base 1002.

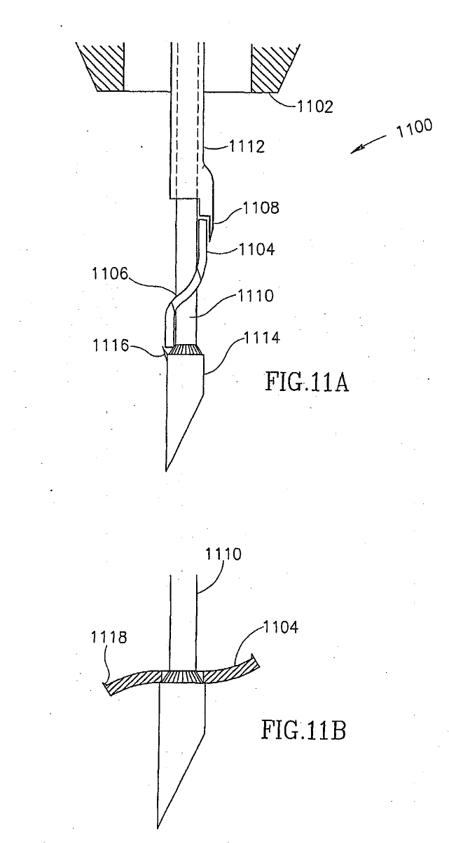

FIGS. 11A and 11B illustrate a hole former 1100 including a geometry changing anvil 1104, in accordance with an exemplary embodiment of the invention. Hole former 1100 includes a penetration tip 1114 mounted on a shaft 1110 and a base tube 1102. A cut-assisting disk 1104, optionally having an aperture 1106 is mounted on shaft 1110. In an exemplary embodiment of the invention, an over tube 1112 (or other similar restraining element) maintains disk 1104 in a distorted configuration, for example, the disk being held between an extension 1108 of tube 1112 and shaft 1110. Optionally, a second extension 1116, holds another portion of disk 1104 against penetration tip 1114.

In FIG. 11B, penetration tip 1114 and disk 1104 are inserted through a blood vessel wall and tube 1112 is retracted, thus freeing disk 1104 to achieve an orientation perpendicular to shaft 1110. Disk 1104 can now be used as an anvil or as a shearing base, depending, inter alia, on the relative geometries of disk 1104 and base 1102. Optionally, disk 1104 includes one or more spikes or a cutting edge 1118, so that it can be used for cutting. Optionally, aperture 1106 of disk 1110 has a geometry that mates the cross-section of shaft 1110, preventing rotation.

In an exemplary embodiment of the invention, disk 1104 is aligned with a direction of a cut formed by penetration tip 1114. Alternatively or additionally, disk 1104 has a sharp edge that assist in forming a cut.

Optionally, disk 1104 is made oblique by the distortion, so that its trans-axial dimension is small. Alternatively or additionally, disk 1104 is always oblique. Alternatively or additionally, disk 1104 is maintained in a distorted configuration by tension, between one part that is held by the penetration tip 1114 and another part that is held back by over tube 1112.

Alternatively or additionally, disk 1104 is plastically distorted, for example, by the advance of over tube 1112 flattening disk 1104. Alternatively or additionally, disk 1104 is bistable between the configurations of FIGS. 11A and 11B.

In this and in other embodiments, various shape changing mechanisms may be used, for example, the above mentioned shape changing mechanism and elastic, super-elastic and shape-memory based distortion.

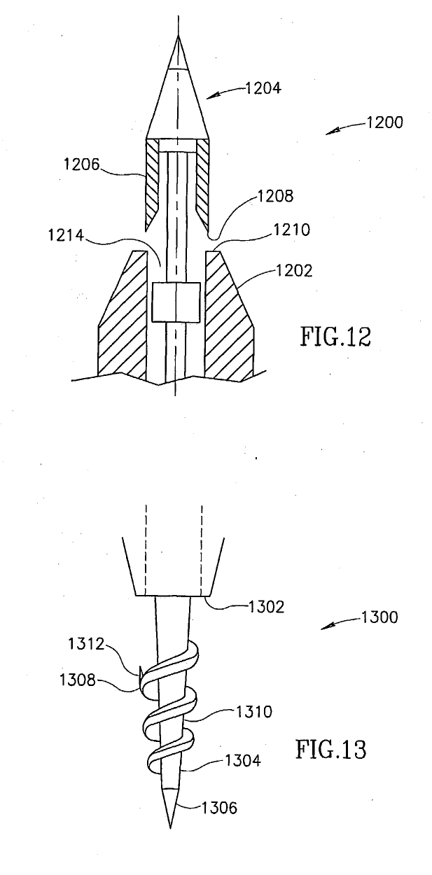

FIG. 12 illustrates a resilient anvil hole former 1200, in accordance with an exemplary embodiment of the invention. Former 1200 comprises a penetration head 1204, for example as described above, which includes a wall 1206 having a cutting edge 1208. A base 1202 is also provided, however, unlike some of the embodiments described above, base 1202 has a front end 1210 that is resilient. In one embodiment, cutting edge 1208 can penetrate into front end 1210. In another embodiment, cutting edge 1208 compresses end 1210 and then optionally slides into an hollow axis 1214 defined by the distorted base 1202. Optionally, the degree of resilience is selected to assist in cutting adventitsia tissue.

FIG. 13 illustrates a hole former 1300 including a thread-type penetration head 1304, in accordance with an exemplary embodiment of the invention. Head 1304 comprises a shaft 1310 on which a threading 1308 is provided. Optionally, a retractable penetration tip 1306 is provided. In use, shaft 1310 is inserted through a blood vessel wall and then rotated to advance the shaft using the threading. Once some or all the threading is through the wall, penetration head 1304 is retracted towards a base 1302, to cut the wall tissue. In one example a cutting edge 1312 is provided on thread 1308. Alternatively or additionally, a shearing cutting action is performed between a thread turn and base 1302.

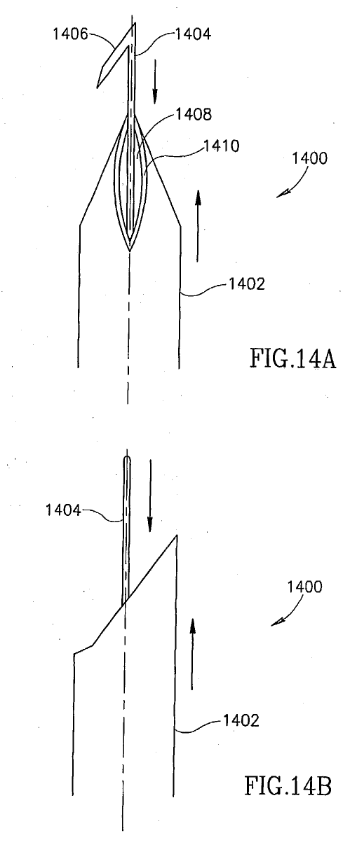

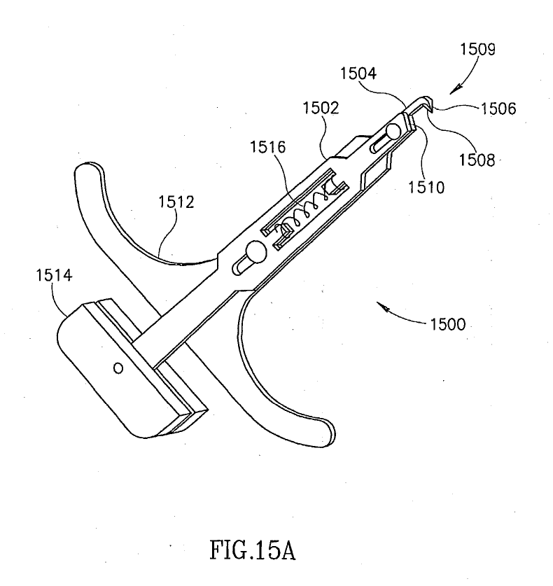

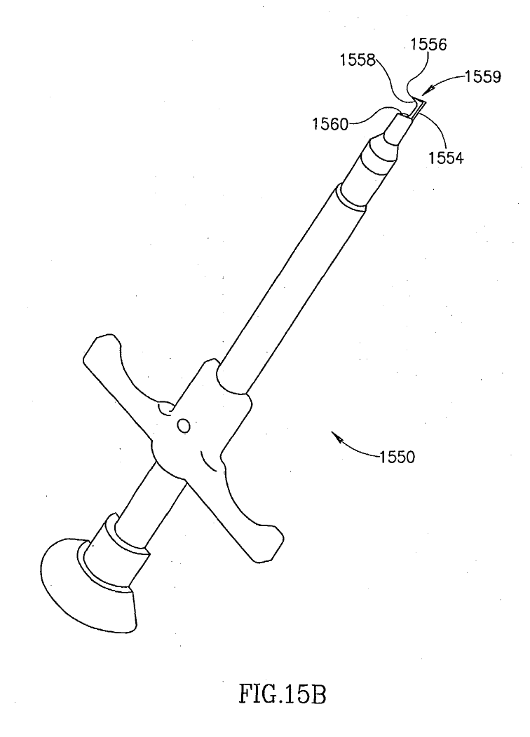

FIGS. 14A and 14B are perpendicular side views of a needle-type hole former 1400, in accordance with an exemplary embodiment of the invention. A hollow pointed needle 1402 is formed with an oblique aperture 1408 optionally having a sharpened cutting lip 1410. In use, a penetration tip 1404 is extended through a wall of a blood vessel and then retracted towards the needle. In an exemplary embodiment of the invention, tip 1404 includes an extension 1406, for example an elastically extending extension that extends once the penetration tip passes out of the needle and through the tissue. Optionally, extension 1406 serves as a knife. Alternatively or additionally, the tip of extension 1406 is inserted into the target blood vessel first and then turned, for example as in the embodiment of FIG. 15.

FIGS. 15A and 15B illustrate two variants of an incision maker, in accordance with an exemplary embodiment of the invention. FIG. 15A shows an incision maker 1500. Two moving parts are provided, a base face 1510 coupled to a first handle 1514 and an “L” shaped spike 1504 coupled to a second handle 1512. Other handle designs may be used. The two parts are optionally coupled using a spring 1516. In use, a tip 1506 of an arm 1509 of spike 1504 is inserted into a blood vessel, for example a coronary artery. Incision maker 1500 is then turned so that arm 1509 is inside the vessel and parallel to the vessel axis (assuming that is the desired cut direction, as an oblique cut or a trans-axial cut may be desired). Arm 1509 is then retracted towards face 1510 and the vessel wall is cut using a shearing cut. Optionally an inner face 1508 of arm 1509 is sharp and functions as a knife.

FIG. 15B shows an alternative embodiment of an incision maker in accordance with the invention, in which two base faces 1560 are provided, one on either side of a spike 1554 (only one face is visible). A spike tip 1556 of an arm 1559 and an optionally cutting edge 1558 of arm 1159 may function as before.

Optionally, face 1560 and arm 1559 while optionally in substantially parallel are not parallel to each other, for example, spreading out (as shown) or pointing in.

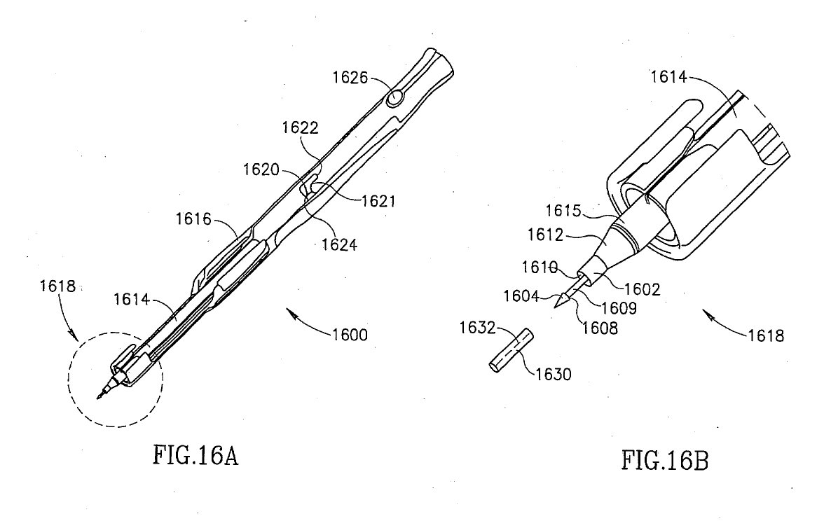

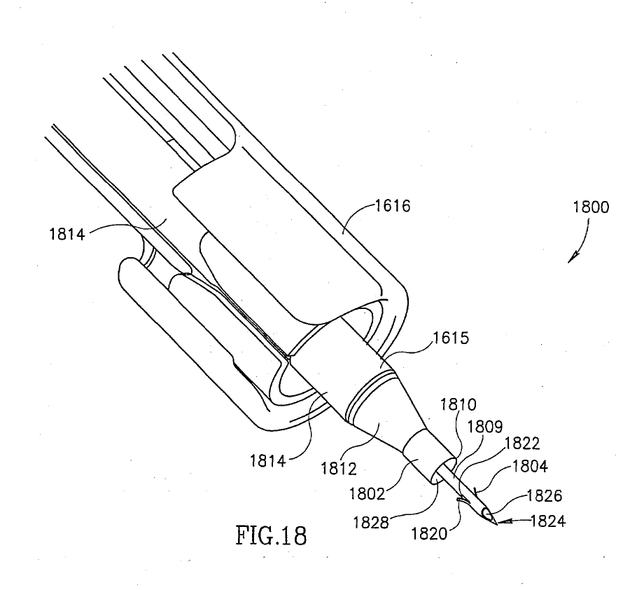

FIGS. 16A and 16B illustrate a hole former system 1600 in accordance with an alternative embodiment of the invention. FIG. 16A shows former 1600 in a scaffold delivery system 1616 and FIG. 16B shows an enlargement of a tip 1618 of former 1600. Referring first to FIG. 16B, former tip 1618 comprises a sharp penetration head 1604 adapted to be inserted into a blood vessel, so that a shaft portion 1609 of penetration head 1604 transfixes the blood vessel wall. Optionally, head 1604 includes a roughened surface, barbs, threads, a tissue receptacle (e.g., 116 of FIG. 1) or a widening 1608 (such as the cone shape shown), to prevent tissue from falling off head shaft 1609, as described in more detail below. In an exemplary embodiment of the invention, angled extensions are formed out of a straight shaft by cutting into the shaft at an angle at several locations (e.g., 2 or 3) and pulling or curling the cut sections out in a radial direction, for example as shown in FIG. 18 below.

Cutting of the target vessel is achieved by a cutting surface 1610 formed on a base section 1602, for example a tube. As noted above, the cutting surface may be smooth, jagged, serrated and/or wave-like, possibly different finishes on different parts of the surface. Optionally, cutting surface 1610 defines an oblique surface relative to shaft 1609 or is not all in one plane. Base 1602 is optionally connected to a shaft 1614 of former 1600, using an inclined section 1612, which may be used for assisting in advancing a sleeve 1615 of scaffold 1616 into a formed aperture in a blood vessel.

Optionally, penetration head 1604 is locked to base section 1602, during cutting, to prevent its axial motion and optionally also its rotational motion.

In an exemplary embodiment of the invention, after a hole is cut using surface 1610, penetration head 1604 is retracted pulling a plug of tissue that is cut out into a lumen in base 1602. Optionally, the retraction is manual. Alternatively, the retraction is spring loaded. Alternatively, other power sources may be used for retraction, for example, pneumatic power, such as available at gas pressure outlets in many hospital rooms. In another example, an electrical motor or solenoid is used to retract penetration head 1604. The retraction may be wholly axial or it may include a rotational component. In some embodiments of the invention, penetration head 1604 has rotational freedom relative to base 1602, while in other embodiments it is rotationally fixed. Base 1602 may or may not rotate relative to scaffold 1616.

In an exemplary embodiment of the invention, a peg 1620 is provided in a channel 1621 which has two resting spots, the position of peg 1620 as shown in FIG. 16A (1624), where head 1604 is extended and a position 1622 at which head 1604 is retracted. Optionally, a safety release switch 1626 is provided to lock head 1604 and prevent axial motion of head 1604 relative to base 1602 and/or to lock the hole former 1600 in delivery scaffold 1616.

The use of a general scaffold 1616 with which different tools can be delivered is not crucial for carrying out the invention. However, some types of such scaffolds include an inner leaflet valve through which the tools are advanced. In some cases, surface 1610 and/or head 1604 may damage the valve when the hold former is advanced through the scaffold. In an exemplary embodiment of the invention, a protective covering 1630 is provided. In an exemplary embodiment of the invention, covering 1630 comprises a tube, for example, a silicone tube or a shrink-fitted tube that isolates the valve from the sharp edges of former 1600 (or other tool), for example, surface 1610 and the tip of head 1604. After insertion, covering 1630 is torn off or pulled off (e.g., if it has one sealed end. Optionally, covering 1630 includes a perforation 1632, a rip cord and/or a pull tab, to assist in removal after it is inserted in scaffold 1616.

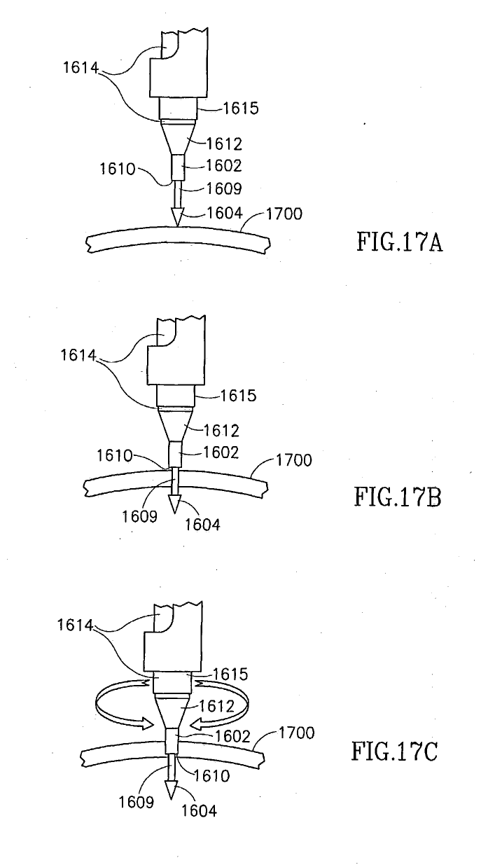

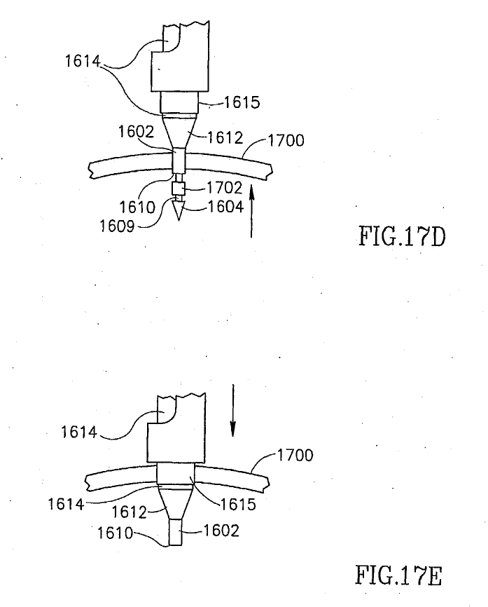

FIGS. 17A-17E illustrate the use of hole former 1600, in accordance with an exemplary embodiment of the invention.

In FIG. 17A, penetration head 1604 is advanced towards a blood vessel, for example an aorta 1700.

In FIG. 17B, penetration head 1604 is advanced to penetrate vessel 1700, so that shaft 1609 transfixes vessel 1700 and penetration head 1604 does not engage vessel 1700 in any way. In some embodiments, however, penetration head 1604 includes barbs for engaging vessel 1700 or remains inside the wall of the vessel. Such engagement may cause the vascular tissue to be stretched before being cut, possibly providing apertures that are smaller or larger than the diameter of base 1602 and/or have a conical profile. The size and shape may depend on whether penetration head 1604 is retracted prior to cutting starting and/or being completed. Optionally, penetration head 1604 includes a retracting sharp tip (e.g., FIG. 4A).

In FIG. 17C, cutting is performed, for example, by rotating and/or advancing base 1602 relative to vessel 1700, so that cutting surface 1610 cuts into vessel 1700. Depending on the implementation of former 1600, the entire delivery system may be moved/rotated or only base 1610 and/or other sub-components of system 1600 are rotated and/or moved.

In FIG. 17D, cutting is complete, so base 1610 is engaged by vessel 1700, while a plug 1702 of tissue remains on shaft 1609. Possibly, some or all of plug 1702 is contained inside base 1602. Optionally, a tissue receptacle (not shown) is provided on penetration head 1604.

Penetration head 1604 is retracted, pulling along with it plug 1702, into a lumen formed in base 1602. Penetration head 1604 optionally has significant clearance relative to the inner diameter of the lumen. Alternatively, a small clearance is provided, so that base 1602 and penetration head 1604 can exhibit a shearing action between them (e.g., to cut any loose strands). Optionally, penetration head 1604 is retracted prior to the cutting being completed, but in a the embodiment pictured, it is not so retracted. Alternatively, penetration head 1604 is retracted while base 1602 is advanced, for example to ensure that it does not damage the far side of the blood vessel. Optionally, however, penetration head 1604 is retracted in a manner that ensures that penetration head 1604 does not apply tension or undue tension on vessel 1700, and affect the aperture cutting shape. In one example the penetration head is retracted such that the distance between penetration head 1604 and base 1602 is greater than the thickness of plug 1702, or at least an uncut thickness thereof.

It should be noted that if vessel 1700 is filled with blood under pressure, there is little danger of penetration head 1604 damaging the far side of vessel 1700, especially if the length of shaft 1609 and penetration head 1604 is considerably less than the diameter of vessel 1700. Alternatively, a retracting penetration tip is provided. Desirably surface 1610 is advanced under light pressure, possibly under its own weight, to prevent distortion of vessel 1700. Alternatively, vessel 1700 may be kept in shape by pressure (e.g., with fingers or a tool) on its sides that are perpendicular to the penetration.

In FIG. 17E, the entire hole former is advanced, so that sleeve 1615 enters the wall of vessel 1700 and the hole forming mechanism can be removed. An anastomosis delivery system may now be provided through scaffold 1616 and its valve.

In an exemplary embodiment of the invention, shaft 1609 has a length greater than the thickness of the wall of vessel 1700, for example, being 150%, 200% or 300% its thickness. In an aorta, this translates, for example, into a length of 4-6 mm. Alternatively, the shaft may be shorter than a vessel diameter. Optionally, different length shafts are provided for different patients and/or vessel sizes. Alternatively, a screw or other mechanism is used to adjust the length of shaft 1606, for example, by controlling the resting location of peg 1620. The diameter of penetration head 1604 may be selected to be the diameter that prevent sliding off of plug 1702, while allowing clearance relative to base 1602. The relation between the diameter of shaft 1609 and cutting surface 1610 is optionally as defined in FIG. 3.

FIG. 18 illustrates a tip of a hole former 1800 in accordance with an alternative embodiment of the invention. Former 1800 comprises a shaft 1814 coupled by a cone 1812 to a base section 1802 having a cutting lip 1810 and an inner lumen having a surface 1828. A penetration head 1804 comprises a needle like shaft 1809 having formed out of its body one or more barbs 1820, cut out of depressions 1822. Other methods of forming and attaching such barbs may be used as well. Optionally, shaft 1809 has a needle like tip 1824 with an optional inner lumen having an inner surface 1826.

In an exemplary embodiment of the invention, barbs 1820 are elastic, so that when inserting head 1804 into vessel 1700, barbs 1820 bend back into depressions 1822 and present a smaller resistance to insertion. After insertion, the spring out again.

Optionally, surface 1826 and/or surface 1828 have inner threads, barbs or other treatment, to better engage tissue plugs. Alternatively, the inner diameter of the lumens vary, for example, non-monotonicly, or monotonicly increasing (away form the blood vessel).

A hollow tip such as provided in FIG. 18 may have other uses as well, for example, for eluting medication (e.g., against clotting, for healing the cut tissue and/or to assist in cutting), for example, continuously or when a suitable control (e.g., attached to a reservoir) is used. Alternatively or additionally, such a lumen is used for providing vacuum to better couple former 1800 and vessel 1700. Alternatively or additionally, vacuum is provided between penetration head 1804 (if any) and base 1802, e.g., through the lumen in base 1802. Alternatively, eluting of medication may be provided in other ways, for example, by penetration head 1804 being spongy or from base 1802, for example, from its lumen or its walls.

In an alternative embodiment of the invention, no penetration head is provided, with tissue plug 1702 optionally prevented from falling off by inner threading of surface 1828 of base 1802. Optionally, however, an axial stabilizer like penetration head 1804 and shaft 1809 are provided. In one example, a wire is provided. Alternatively, a spiral cork-screw like shaft 1809 is provided. This inner stabilizer may or may not have a fixed axial position relative to base 1802. If not fixed, the range of motion may nevertheless be fixed and/or the number of stable positions be limited. In an exemplary embodiment of the invention, the stabilizer is fixed so that it protrudes by a large amount (e.g., 1-5 mm for an aorta), slightly (e.g., 1 mm), is even with or is retracted relative to a plane defined by surface 1810. Optionally, the stabilizer is not strong enough (or does not engage vessel 1700 well enough) to be used to urge vessel 1700 against base 1802.

The above description has focused on devices that are applied from outside a blood vessel. However, they can also be applied from inside of blood vessels.

In an exemplary embodiment of the invention, the design is optionally changed to accommodate one or more of the following factors:

(a) which layer of the blood vessel is to be cut more precisely;

(b) what type of cutting action to apply to each blood vessel layer;

(c) disposal of the tissue plug (if any) to outside the blood vessel or to inside the delivery system; and/or desired cut profile.

In one example of an inside-out punch, the tissue receptacle is located on the base and has a cutting lip that extends forward. In another example, the tissue receptacle is on the penetration head but the base advances forwards towards the receptacle.

In addition, the aperture forming systems may be provided in several sizes, for example, two, three or more sizes.

It should be noted that the elements described as tubes are not generally required to be tubes. In one example, the apertured base tube can be replaced by a slotted solid rod, in which the slot carries a shaft for retraction of the penetration head. The shaft need not attach to the center of the penetration head.

It should also be noted that hole formers can be used to create incomplete removal of plugs, for example, to create rectangular or triangular flaps.

In an exemplary embodiment of the invention, the above devices are used in combination with anastomosis-related tools as described in PCT applications and publications WO 99/62415, WO 00/56226, WO 00/56228, WO 01/41623, WO 01/41624, PCT/IL01/00267, PCT/IL01/00069, PCT/IL01/00074, and PCT/IL01/00266, the disclosures of which are incorporated herein by reference. However, they may also be used as stand alone devices or as part of surgical kits for other uses and/or anastomosis connectors.

It will be appreciated that the above described methods and devices of vascular manipulation may be varied in many ways, including, changing the order of steps, the exact materials used for the devices, which vessel is a “side” side and which vessel (or graft) is an “end” side of an end-to-side anastomosis. Further, in the mechanical embodiments, the location of various elements may be switched, without exceeding the spirit of the disclosure, for example, switching the moving elements for non-moving elements where relative motion is required. In addition, a multiplicity of various features, both of methods and of devices have been described. It should be appreciated that different features may be combined in different ways. In particular, not all the features shown above in a particular embodiment are necessary in every similar exemplary embodiment of the invention. Further, combinations of the above features, from different described embodiments are also considered to be within the scope of some exemplary embodiments of the invention. In addition, some of the features of the invention described herein may be adapted for use with prior art devices, in accordance with other exemplary embodiments of the invention. The particular geometric forms used to illustrate the invention should not be considered as necessarily limiting the invention in its broadest aspect to only those forms, for example, where a circular lumen is shown, in other embodiments an oval lumen may be used.

Also within the scope of the invention are surgical kits which include sets of medical devices suitable for making a single or a small number of anastomosis connections and/or apertures. Measurements are provided to serve only as exemplary measurements for particular cases, the exact measurements applied will vary depending on the application. When used in the following claims, the terms “comprises”, “comprising”, “includes”, “including” or the like means “including but not limited to”.

It will be appreciated by a person skilled in the art that the present invention is not limited by what has thus far been described. Rather, the scope of the present invention is limited only by the following claims.

1. Hole forming apparatus for forming an opening in a blood vessel, comprising:
a penetration head having a sharp tip adapted to be inserted through a wall of a blood vessel;
a base section defining an inner lumen;
at least one cutting surface defined on said base section and adapted to cut through a wall of said blood vessel without said apparatus pulling said blood vessel towards said cutting surface; and
a shaft mechanically coupling said penetration head to said base section,
said shaft defining a tissue holding shaft section between said base section and said penetration head, said penetration head adapted to prevent tissue transfixed on said shaft section from slipping off said penetration head,
said shaft having a first mechanically defined and axially locked resting position in which said penetration head is at least partially enclosed by said base section in said inner lumen and a second mechanically defined resting position in which said penetration head is axially locked relative to said base section and said shaft section is exposed between said base section and said penetration head.

2. Apparatus according to claim 1, wherein moving said shaft between said positions is not mechanically coupled to rotation of said base section.

3. Apparatus according to claim 1, wherein said penetration head has a geometry of a cone.

4. Apparatus according to claim 1, wherein said penetration head comprises at least one protrusion having a radial extent greater than a minimum diameter of said shaft section.

5. Apparatus according to claim 4, wherein said at least one protrusion comprises a barb cut out of said shaft section.

6. Apparatus according to claim 5, wherein said barb is elastic and flexible enough to be pushed against said shaft section by said wall of said vessel when said penetration head is inserted into said vessel.

7. Apparatus according to claim 4, wherein said penetration head comprises a retractable penetration tip.

8. Apparatus according to claim 1, wherein said penetration head is rotationally locked relative to said base section.

9. Apparatus according to claim 1, wherein in said first resting position said penetration head is fully retracted into said lumen.

10. Apparatus according to claim 1, wherein said shaft section is at least 150% greater than a width of a vessel for which the apparatus is designed.

11. Apparatus according to claim 1, adapted for forming an aperture from outside a blood vessel.

12. Apparatus according to claim 1, wherein said shaft is spring loaded to move from said first resting position to said second resting position, when released.

13. Apparatus according to claim 1, wherein a motion of said shaft relative to said base is restricted to be between said two positions.

14. Apparatus according to claim 1, wherein said shaft has only two resting positions.

15. Apparatus according to claim 1, wherein said shaft is rigid.

16. Apparatus according to claim 1, wherein said inner lumen has a clearance relative to said penetration head such that no vascular tissue is cut between said penetration head and said inner lumen.

17. Apparatus according to claim 1, wherein said penetration head is adapted and arranged to not pull back said vessel wall during said cutting.

18. Apparatus according to claim 1, wherein said penetration head defines a lumen in the direction of said wall.

19. Apparatus according to claim 1, comprising externally powered means from moving said shaft between said positions.

20. Apparatus according to claim 1, comprising a pharmaceutical source coupled to an opening defined adjacent said penetration head and said base.

21. Hole forming apparatus for forming an opening in a blood vessel, comprising:
a penetration head having a sharp tip adapted to be inserted through a wall of a blood vessel, said tip defining a lumen in a direction of said wall;
a base section defining an inner lumen;
at least one cutting surface defined on said base section and adapted to cut through a wall of said blood vessel; and
a shaft mechanically coupling said penetration head to said base section and fixing an axial position of said penetration head relative to said base.

22. Apparatus according to claim 21, wherein said lumen elutes a pharmaceutical.

23. Apparatus according to claim 21, wherein said lumen is adapted to engage tissue.

24. Apparatus according to claim 21, wherein said lumen is adapted to allow one directional of motion of tissue relative to the lumen.

25. Hole forming apparatus for forming an opening in a blood vessel, comprising:
a penetration head having a sharp tip adapted to be inserted through a wall of a blood vessel;
a base section defining an inner lumen;
at least one cutting surface defined on said base section and adapted to cut through a wall of said blood vessel without said apparatus pulling said blood vessel towards said cutting surface; and
a shaft mechanically coupling said penetration head to said base section and fixing an axial position of said penetration head relative to said base.

26. Apparatus according to claim 25, wherein said inner lumen is adapted to engage tissue.

27. Apparatus according to claim 25, wherein said penetration head has a fixed diameter.

28. A method of inserting a tool into a scaffold having an inner valve, comprising:
covering at least one sharp edge of said tool with a cover;
inserting said tool through said valve; and
removing said cover.

29. A method according to claim 28, wherein said removing comprises tearing.

30. Hole forming apparatus for forming an opening in a blood vessel, comprising:
a base section defining an inner lumen, said inner lumen being adapted to engage blood vessel tissue; and
at least one cutting surface defined on said base section and adapted to cut through a wall of said blood vessel.

31. Apparatus according to claim 30, wherein said adaptation comprises an inner threading.

32. Apparatus according to claim 30, comprising a trans-axial stabilizer having a fixed axial position relative to said cutting surface.

33. Hole forming apparatus for forming an opening in a blood vessel, comprising:
means for forming an aperture in a blood vessel; and
eluting means for providing a pharmaceutical at said aperture.

34. Apparatus according to claim 33, wherein said means for forming comprises a cutting means.

35. Apparatus according to claim 33, wherein said means for forming comprises a shearing means.

36. Apparatus according to claim 33, wherein said means for forming comprises anvil cutting means.

37. A method of forming an aperture in a blood vessel, comprising:
inserting a penetration head into a wall of a blood vessel; and
advancing a cutting base against said blood vessel while not applying a contra force to said blood vessel via said penetration head.

38. A method according to claim 37, wherein advancing comprises advancing using rotational motion.
